(19) United States
(12) Patent Application Publication
(10) Pub. No.: US 2025/0262440 A1
(43) Pub. Date: Aug. 21, 2025

(54) SYSTEMS AND METHODS FOR PROVIDING DYNAMIC NEUROSTIMULATION

(71) Applicant: Boston Scientific Neuromodulation Corporation, Valencia, CA (US)

(72) Inventors: Changfang Zhu, Valencia, CA (US); Rosana Esteller, Santa Clarita, CA (US)

(21) Appl. No.: 19/049,888

(22) Filed: Feb. 10, 2025

Related U.S. Application Data
(60) Provisional application No. 63/555,347, filed on Feb. 19, 2024.

(51) Int. Cl. A61N 1/36

(52) U.S. Cl. CPC A61N 1/36178; A61N 1/36021; A61N 1/36164; A61N 1/36171; A61N 1/36175

(57) ABSTRACT

A system may include an electrode set including at least one electrode, and a neurostimulator. The neurostimulator may include a tonic waveform generator, a neurostimulation modifier, and a neurostimulator output. The tonic waveform generator may be configured to generate a train of tonic pulses. The tonic pulses may have a uniform pulse-to-pulse interval. The neurostimulation modifier may be configured to apply a neurostimulation-effect modulation to modify the train of tonic pulses. The neurostimulator output may be configured to use the modified train of pulses to deliver a sequence of electrical pulses to a neural target using the electrode set. The neurostimulation modifier may be configured to apply the neurostimulation-effect modulation to change at least one stimulation parameter for at least a portion of the train of tonic pulses to enhance or reduce a neurostimulation effect for one or more pulses within the sequence of electrical pulses.

### CLAIM OF PRIORITY

This application claims the benefit of U.S. Provisional Application No. 63/555,347, filed on Feb. 19, 2024, which is hereby incorporated by reference in its entirety.

### TECHNICAL FIELD

This document relates generally to medical systems, and more particularly, but not by way of limitation, to systems, devices, and methods for providing neurostimulation.

### BACKGROUND

Neurostimulation has been proposed as a therapy for a number of conditions. Often, neural modulation and neural stimulation may be used interchangeably to describe excitatory stimulation that causes action potentials as well as inhibitory and other effects. In an effort to avoid confusion over the different uses of the term “modulation,” the present application uses the term “stimulation” to discuss a neurostimulation energy signal delivered through electrode(s) to the patient, and uses the term “modulation” to for modulating or adjusting the signal. Examples of neurostimulation include Spinal Cord Stimulation (SCS), Deep Brain Stimulation (DBS), Peripheral Nerve Stimulation (PNS), and Functional Electrical Stimulation (FES). SCS, by way of example and not limitation, has been used to treat chronic pain syndromes.

An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

The neurostimulation energy may be delivered using a waveform in the form of electrical neurostimulation pulses. Neurostimulation systems may be programmed with pulse patterns that are either tonic in nature (i.e., a continuous pulse pattern having a uniform pulse rate, pulse width, pulse amplitude, etc. that predictably generates action potentials in the nervous system) or bursted in nature (i.e., a pulse pattern that is alternately turned on and off). However, human nervous systems use neural signals having much more sophisticated patterns to communicate various types of information, including sensations of pain, pressure, temperature, etc. The nervous system may interpret an artificial stimulation with a simple pattern of stimuli as an unnatural phenomenon, and respond with an unintended and undesirable sensation and/or movement. For example, some neurostimulation therapies are known to cause paresthesia and/or vibration of non-targeted tissue or organ.

The efficacy and efficiency of certain neurostimulation therapies may be improved, and their side-effects may be reduced, by using patterns of neurostimulation pulses that emulate natural patterns of neural signals observed in the human body. The effectiveness of a neurostimulation regimen may include a spatial component (i.e., stimulating in the right location which is highly dependent on the disorder to be treated), a temporal component (i.e., stimulating responsive to state such as sensing a relevant physiological parameter and responding with stimulation), and an informational component (i.e., pulsing with patterns to send the appropriate information to stimulate).

Modern electronics can accommodate the need for generating sophisticated signal patterns. However, the capability of a neurostimulation system depends on how stimulation parameters defining such a signal pattern can be generated in an efficient manner for programming a stimulation. For example, a customized neurostimulation system may be limited to that particular application and generally not usable for applications using different non-tonic pulse patterns.

It is desirable to provide a neurostimulation system that can provide dynamic neurostimulation. As it is relatively easy to generate tonic pulses, it is desirable to be able to efficiently produce a customized dynamic stimulation effect from tonic pulses.

### SUMMARY

By way of example and not limitation, various embodiments provide dynamic pulse modulation by modulating at least one parameter of a tonic pulse train, and various embodiments provide hybrid pulse modulation by modulating more than one stimulation parameter at a time. Furthermore, more than one stimulation parameter may be modulated at a time to provide dynamic pulse modulation. This modulation of more than one stimulation parameter at a time may be termed hybrid pulse modulation.

An example (e.g., Example 1) of a system may include an electrode set including at least one electrode, and a neurostimulator. The neurostimulator may include a tonic waveform generator, a neurostimulation modifier, and a neurostimulator output. The tonic waveform generator may be configured to generate a train of tonic pulses. The tonic pulses may have a uniform pulse-to-pulse interval. The neurostimulation modifier may be configured to apply a neurostimulation-effect modulation to modify the train of tonic pulses. The neurostimulator output may be configured to use the modified train of pulses to deliver a sequence of electrical pulses to a neural target using the electrode set. The neurostimulation modifier may be configured to apply the neurostimulation-effect modulation to change at least one stimulation parameter for at least a portion of the train of tonic pulses to enhance or reduce a neurostimulation effect for one or more pulses within the sequence of electrical pulses.

In Example 2, the subject matter of Example 1 may optionally be configured such that the neurostimulator output includes a driver configured to use the modified pulse train to drive the sequence of electrical pulses to the neural target using the electrode set.

In Example 3, the subject matter of Example 2 may optionally be configured such that the driver includes energy sources, and the neurostimulation modifier is implemented using firmware to adjust energy sources. Examples of energy sources may include current sources, voltage sources and/or energy storage units.

In Example 4, the subject matter of Example 2 may optionally be configured such that the neurostimulation modifier includes hardware circuits configured to modulate the train of tonic pulses using the neurostimulation-effect modulation.

In Example 5, the subject matter of Example 2 may optionally be configured such that the tonic waveform generator and the neurostimulation modifier are implemented using software, and both the train of tonic pulses and the modified train of pulses are digital.

In Example 6, the subject matter of any one or more of Examples 1-5 may optionally be configured such that the neurostimulation-effect modulation is a charge modulation function.

In Example 7, the subject matter of Example 6 may optionally be configured such that the neurostimulator output is configured to apply the charge modulation function by modulating more than one stimulation parameter for at least the portion of the train of tonic pulses.

In Example 8, the subject matter of Example 7 may optionally be configured such that the neurostimulator output is configured to apply the charge modulation function by modulating at least two stimulation parameters from: amplitude, pulse width, pulse frequency, a charge balance configuration, burst duration, burst frequency or burst ON/OFF duty cycle.

In Example 9, the subject matter of any one or more of Examples 1-8 may optionally be configured such that the neurostimulation-effect modulation is user-programmable.

In Example 10, the subject matter of any one or more of Examples 1-8 may optionally be configured such that the neurostimulation-effect modulation is a predetermined function programmed in the system.

In Example 11, the subject matter of any one or more of Examples 1-10 may optionally be configured such that the sequence of electrical pulses includes a section of monophasic pulses and a recharge balance section for balancing delivered net charge.

In Example 12, the subject matter of any one or more of Examples 1-10 may optionally be configured such that the sequence of electrical pulses includes two or more phases.

In Example 13, the subject matter of Example 12 may optionally be configured such that the system is configured to apply the neurostimulation-effect modulation by providing a first value for at least one parameter in a first phase independent of a second value for the at least one parameter in a second phase.

In Example 14, the subject matter of any one or more of Examples 1-13 may optionally be configured such that the neurostimulator output is configured to apply the neurostimulation-effect modulation function to provide some pulses within the electrical pulse energy that engage neural tissue and to provide other pulses within the electrical pulse energy that do not engage neural tissue.

In Example 15, the subject matter of any one or more of Examples 1-13 may optionally be configured such that the neurostimulator output is configured to apply the neurostimulation-effect modulation function to provide some pulses within the electrical pulse energy that are supra-threshold stimulation and to provide other pulses within the electrical pulse energy that are sub-threshold stimulation.

Example 16 includes subject matter (such as a method, means for performing acts, machine readable medium including instructions that when performed by a machine cause the machine to perform acts, or an apparatus to perform). The subject matter may include: generating, using a tonic waveform generator, a train of tonic pulses having a uniform pulse-to-pulse interval; applying, using a neurostimulation modifier, a neurostimulation-effect modulation to modify the train of tonic pulses; and delivering a sequence of electrical pulses to a neural target using an electrode set that includes at least one electrode, wherein the modified train of tonic pulses and a neurostimulator output are used to deliver the sequence. The applied neurostimulation-effect modulation changes at least one stimulation parameter for at least a portion of the train of tonic pulses to enhance or reduce a neurostimulation effect for one or more pulses within the sequence of electrical pulses.

In Example 17, the subject matter of Example 16 may optionally be configured such that the sequence is delivered using a driver to drive the sequence to the neural target.

In Example 18, the subject matter of Example 17 may optionally be configured such that the driver includes energy sources, and wherein the neurostimulation-effect modulation is applied using firmware to adjust energy sources.

In Example 19, the subject matter of Example 17 may optionally be configured such that the neurostimulation-effect modulation is applied using hardware circuits configured to modulate the train of tonic pulses.

In Example 20, the subject matter of Example 17 may optionally be configured such that the tonic waveform generator and the neurostimulation modifier are implemented using software, and both the train of tonic pulses and the modified train of pulses are digital.

In Example 21, the subject matter of any one or more of Examples 16-20 may optionally be configured such that the neurostimulation-effect modulation is a charge modulation function.

In Example 22, the subject matter of Example 21 may optionally be configured such that the charge modulation function is applied by modulating more than one stimulation parameter for at least the portion of the train of tonic pulses.

In Example 23, the subject matter of Example 22 may optionally be configured such that the charge modulation function is applied by modulating at least two stimulation parameters from: amplitude, pulse width, pulse frequency, a charge balance configuration, burst duration, burst frequency or burst ON/OFF duty cycle.

In Example 24, the subject matter of any one or more of Examples 16-23 may optionally be configured to further include receiving a user input to program the neurostimulation-effect modulation.

In Example 25, the subject matter of any one or more of Examples 16-24 may optionally be configured to further include the neurostimulation-effect modulation is a predetermined function in the system that is not user-programmable.

In Example 26, the subject matter of any one or more of Examples 16-25 may optionally be configured such that the sequence of electrical pulses includes a section of monophasic pulses and a recharge balance section for balancing delivered net charge.

In Example 27, the subject matter of any one or more of Examples 16-25 may optionally be configured such that the sequence of electrical pulses includes two or more phases.

In Example 28, the subject matter of Example 27 may optionally be configured such that the neurostimulation-effect modulation is applied by providing a first value for at least one parameter in a first phase independent of a second value for the at least one parameter in a second phase.

In Example 29, the subject matter of any one or more of Examples 16-28 may optionally be configured such that the neurostimulation-effect modulation function is applied to provide some pulses within the electrical pulse energy that engage neural tissue and to provide other pulses within the electrical pulse energy that do not engage neural tissue.

In Example 30, the subject matter of any one or more of Examples 16-28 may optionally be configured such that the neurostimulation-effect modulation function is applied to provide some pulses within the electrical pulse energy that are supra-threshold stimulation and to provide other pulses within the electrical pulse energy that are sub-threshold stimulation.

Example 31 includes subject matter (such as a non-transitory machine-readable medium including instructions, which when executed by a machine, cause the machine to perform a method including generating, using a tonic waveform generator, a train of tonic pulses having a uniform pulse-to-pulse interval; applying, using a neurostimulation modifier, a neurostimulation-effect modulation to modify the train of tonic pulses; and delivering a sequence of electrical pulses to a neural target using an electrode set that includes at least one electrode, wherein the modified train of tonic pulses and a neurostimulator output are used to deliver the sequence. The applied neurostimulation-effect modulation changes at least one stimulation parameter for at least a portion of the train of tonic pulses to enhance or reduce a neurostimulation effect for one or more pulses within the sequence of electrical pulses. In additional examples, the subject matter of Examples 17-30 may be performed by the machine.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an embodiment of a neurostimulation system.

FIG. 2 illustrates an embodiment of a neurostimulator, such as may be implemented in the neurostimulation system of FIG. 1.

FIG. 3 illustrates an embodiment of a programming system such as a programming device, which may be implemented as the programming device in the neurostimulation system of FIG. 1.

FIG. 4 illustrates, by way of example, an implantable neurostimulation system and portions of an environment in which system may be used.

FIG. 5 illustrates, by way of example, an embodiment of a SCS system, which also may be referred to as a Spinal Cord Modulation (SCM) system.

FIG. 6 illustrates, by way of example and not limitation, a neurostimulator embodiment including a tonic waveform generator, a neurostimulation modifier, and a neurostimulator output.

FIG. 7 illustrates, by way of example and not limitation, a method for modifying a train of tonic pulses.

FIG. 8 illustrates, by way of example and not limitation, a tonic waveform, a modulation waveform to modulate the tonic waveform, and a modulated tonic waveform to provide a sequence of electrical pulses.

FIG. 9 illustrates, by way of example and not limitation, a neurostimulation effect modifier and corresponding modulator which may be a specific embodiment of the neurostimulation effect modifier and modulator illustrated in FIG. 6.

FIG. 10 illustrates, by way of example and not limitation, a neurostimulator embodiment including a tonic waveform generator, a neurostimulation modifier, and current sources.

FIG. 11 illustrates, by way of example and not limitation, a method for modifying a train of tonic pulses.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter provides systems, devices, and methods for providing dynamic neurostimulation. At least one parameter of a tonic pulse train may be modulated to provide dynamic neurostimulation. More than one stimulation parameter may be modulated at a time to provide hybrid pulse modulation.

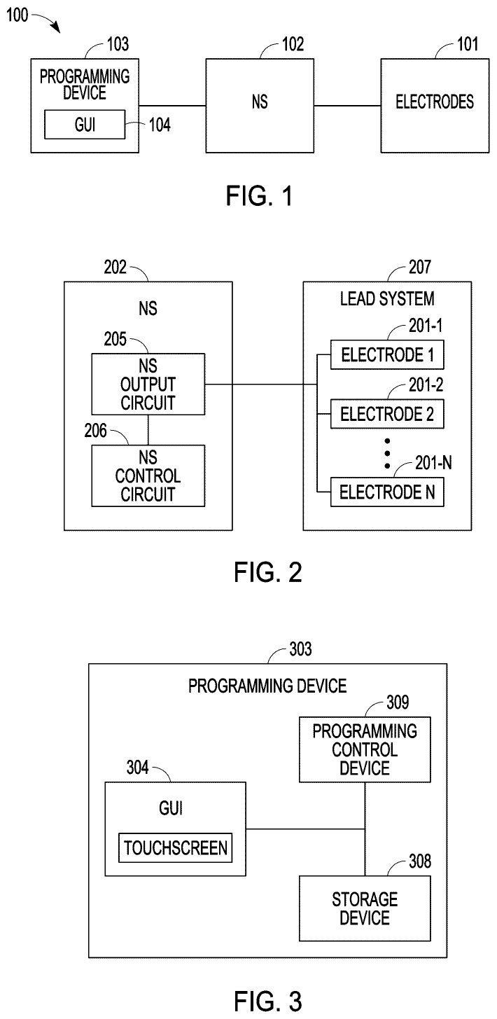

FIG. 1 illustrates, by way of example and not limitation, an embodiment of a neurostimulation system. The illustrated system 100 includes electrode contacts 101, which also may be simply referred to as electrodes, a neurostimulator 102, and a programming system such as a programming device 103. The programming system may include multiple devices. The electrode contacts 101 are configured to be placed on or near one or more neural targets in a patient. The neurostimulator 102 is configured to be electrically connected to electrode contacts 101 and deliver neurostimulation energy, such as in the form of electrical pulses, to the one or more neural targets though electrode contacts 101. The delivery of the neurostimulation is controlled using a plurality of neurostimulation parameters. The neurostimulation parameters may specify the electrical waveform (e.g., pulses or pulse patterns or other waveform shapes) and a selection of electrode contacts through which the electrical waveform is delivered. In various embodiments, at least some parameters of the plurality of neurostimulation parameters are programmable by a user, such as a physician or other caregiver. The programming device 103 provides the user with accessibility to the user-programmable parameters. In various embodiments, the programming device 103 is configured to be communicatively coupled to neurostimulator via a wired or wireless link. In various embodiments, the programming device 103 includes a graphical user interface (GUI) 104 that allows the user to set and/or adjust values of the user-programmable neurostimulation parameters.

FIG. 2 illustrates an embodiment of a neurostimulator 202, such as may be implemented in the neurostimulation system 100 of FIG. 1. Various embodiments of the neurostimulator may be used to deliver different types of neural therapy such as, but not limited to, SCS, DBS, PNS or FES therapy. The illustrated embodiment of the neurostimulator 202 includes a neurostimulation output circuit 205 and a neurostimulation control circuit 206. Those of ordinary skill in the art will understand that the neurostimulation system may include additional components such as sensing circuitry for patient monitoring and/or feedback control of the therapy, telemetry circuitry and power. The neurostimulation output circuit 205 produces and delivers the neurostimulation. Neurostimulation pulses are provided herein as an example. However, the present subject matter is not limited to pulses, but may include other electrical waveforms (e.g., waveforms with different waveform shapes, and waveforms with various pulse patterns). The neurostimulation control circuit 206 controls the delivery of the neurostimulation pulses using the plurality of neurostimulation parameters. The lead system 207 includes one or more leads each configured to be electrically connected to neurostimulator 202 and a plurality of electrode contacts 201-1 to 201-N distributed in an electrode contact arrangement using the one or more leads. Each lead may have an electrode contact array consisting of two or more electrode contacts, which also may be referred to as electrodes. Multiple leads may provide multiple electrode contact arrays to provide the electrode contact arrangement. Each electrode contact is a single electrically conductive contact providing for an electrical interface between neurostimulation output circuit 205 and tissue of the patient, where N≥2. The neurostimulation pulses are each delivered from the neurostimulation output circuit 205 through a set of electrode contacts selected from the electrode contacts 201-1 to 201-N. The number of leads and the number of electrode contacts on each lead may depend on, for example, the distribution of target(s) of the neurostimulation and the need for controlling the distribution of electric field at each target. In one embodiment, by way of example and not limitation, the lead system may include two leads each having eight electrode contacts. Some embodiments may use a lead system that includes a paddle lead.

The actual number and shape of leads and electrode contacts may vary for the intended application. An implantable waveform generator may include an outer case for housing the electronic and other components. The outer case may be composed of an electrically conductive, biocompatible material, such as titanium, that forms a hermetically-sealed compartment wherein the internal electronics are protected from the body tissue and fluids. In some cases, the outer case may serve as an electrode contact (e.g., case electrode). The waveform generator may include electronic components, such as a controller/processor (e.g., a microcontroller), memory, a battery, telemetry circuitry, monitoring circuitry, neurostimulation output circuitry, and other suitable components known to those skilled in the art. The microcontroller executes a suitable program stored in memory, for directing and controlling the neurostimulation performed by the waveform generator. Electrical neurostimulation energy is provided to the electrode contacts in accordance with a set of neurostimulation parameters programmed into the pulse generator. By way of example but not limitation, the electrical neurostimulation energy may be in the form of a pulsed electrical waveform. Such neurostimulation parameters may comprise electrode contact combinations, which define the electrode contacts that are activated as anodes (positive), cathodes (negative), and turned off (zero), percentage of neurostimulation energy assigned to each electrode contact (fractionalized electrode contact configurations), and electrical pulse parameters, which define the pulse amplitude (measured in milliamps or volts depending on whether the pulse generator supplies constant current or constant voltage to the electrode contact array), pulse width (measured in microseconds), pulse rate (measured in pulses per second), and burst rate (measured as the neurostimulation on duration X and neurostimulation off duration Y). Electrode contacts that are selected to transmit or receive electrical energy are referred to herein as “activated,” while electrode contacts that are not selected to transmit or receive electrical energy are referred to herein as “non-activated.”

Electrical neurostimulation occurs between or among a plurality of activated electrode contacts, one of which may be the case of the waveform generator. The system may be capable of transmitting neurostimulation energy to the tissue in a monopolar or multipolar (e.g., bipolar, tripolar, etc.) fashion. Monopolar neurostimulation occurs when a selected one of the lead electrode contacts is activated along with the case of the waveform generator, so that neurostimulation energy is transmitted between the selected electrode contact and case. Any of the electrode contacts E1-E16 and the case electrode contact may be assigned to up to k possible groups or timing “channels.” In one embodiment, k may equal four. The timing channel identifies which electrode contacts are selected to synchronously source or sink current to create an electric field in the tissue to be stimulated. Amplitudes and polarities of electrode contacts on a channel may vary. In particular, the electrode contacts can be selected to be positive (anode, sourcing current), negative (cathode, sinking current), or off (no current) polarity in any of the k timing channels. The waveform generator may be operated in a mode to deliver electrical neurostimulation energy that is therapeutically effective and causes the patient to perceive delivery of the energy (e.g., therapeutically effective to relieve pain with perceived paresthesia), and may be operated in a sub-perception mode to deliver electrical neurostimulation energy that is therapeutically effective and does not cause the patient to perceive delivery of the energy (e.g., therapeutically effective to relieve pain without perceived paresthesia). The waveform generator may also be configured to deliver waveforms or pulses that may be used to prime or condition the tissue for therapy, or waveforms or pulses that are useful for intermittent charge balancing.

The waveform generator may be configured to individually control the magnitude of electrical current flowing through each of the electrode contacts. For example, a current generator may be configured to selectively generate individual current-regulated amplitudes from independent current sources for each electrode contact. In some embodiments, the pulse generator may have voltage regulated outputs. While individually programmable electrode contact amplitudes are desirable to achieve fine control, a single output source switched across electrode contacts may also be used, although with less fine control in programming. Neuromodulators may be designed with mixed current and voltage regulated devices.

The neurostimulation system may be configured to modulate spinal target tissue or other neural tissue. The configuration of electrode contacts used to deliver electrical pulses to the targeted tissue constitutes an electrode contact configuration, with the electrode contacts capable of being selectively programmed to act as anodes (positive), cathodes (negative), or left off (zero). In other words, an electrode contact configuration represents the polarity being positive, negative, or zero. An electrical waveform may be controlled or varied for delivery using electrode contact configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode contact configuration, along with the electrical pulse parameters, can be referred to as a “neurostimulation parameter set.” Each set of neurostimulation parameters, including fractionalized current distribution to the electrode contacts (as percentage cathodic current, percentage anodic current, or off), may be stored and combined into a neurostimulation program that can then be used to modulate multiple regions within the patient.

The number of electrode contacts available combined with the ability to generate a variety of complex electrical waveforms (e.g., pulses), presents a huge selection of neurostimulation parameter sets to the clinician or patient. For example, if the neurostimulation system to be programmed has sixteen electrode contacts, millions of neurostimulation parameter sets may be available for programming into the neurostimulation system. Furthermore, for example SCS systems may have thirty-two electrode contacts which exponentially increases the number of neurostimulation parameters sets available for programming. To facilitate such selection, the clinician generally programs the neurostimulation parameters sets through a computerized programming system to allow the optimum neurostimulation parameters to be determined based on patient feedback, sensor feedback or other means and to subsequently program the desired neurostimulation parameter sets.

FIG. 3 illustrates an embodiment of a programming system such as a programming device 303, which may be implemented as the programming device 103 in the neurostimulation system of FIG. 1. The programming device 303 includes a storage device 308, a programming control circuit 309, and a graphical user interface (GUI) 304. The programming control circuit 309 generates the plurality of neurostimulation parameters that control the delivery of the neurostimulation pulses according to the pattern of the neurostimulation pulses. In various embodiments, the GUI 304 includes any type of presentation device, such as interactive or non-interactive screens, and any type of user input devices that allow the user to program the neurostimulation parameters, such as touchscreen, keyboard, keypad, touchpad, trackball, joystick, and mouse. The storage device 308 may store, among other things, neurostimulation parameters to be programmed into the neurostimulator. Some embodiments may store in the storage device 308 sensed data samples and/or local extrema data for a signal. The programming device 303 may transmit the plurality of neurostimulation parameters to the neurostimulator. In some embodiments, the programming device 303 may transmit power to the neurostimulator. The programming control circuit 309 may generate the plurality of neurostimulation parameters. In various embodiments, the programming control circuit 309 may check values of the plurality of neurostimulation parameters against safety rules to limit these values within constraints of the safety rules.

In various embodiments, circuits of neurostimulation, including its various embodiments discussed in this document, may be implemented using a combination of hardware, software and firmware. For example, the GUI circuit, neurostimulation control circuit, and programming control circuit, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

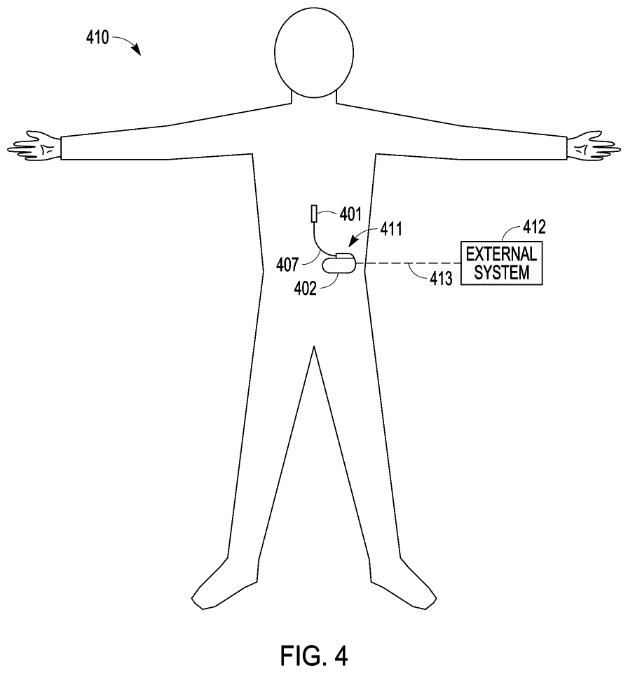

FIG. 4 illustrates, by way of example, an implantable neurostimulation system and portions of an environment in which system may be used. The system is illustrated for implantation near the spinal cord. However, neurostimulation system may be configured to modulate other neural targets including, but not limited to, SBS, PNS or FES targets. The system 410 includes an implantable system 411, an external system 412, and a telemetry link 413 providing for wireless communication between implantable system 411 and external system 412. The implantable system is illustrated as being implanted in the patient's body. The implantable system 411 includes an implantable neurostimulator (also referred to as an implantable pulse generator, or IPG) 402, a lead system 407, and electrode contacts 401. The lead system 407 includes one or more leads each configured to be electrically connected to the neurostimulator 402 and a plurality of electrode contacts 401 distributed in the one or more leads. In various embodiments, the external system 412 includes one or more external (non-implantable) devices each allowing a user (e.g., a clinician or other caregiver and/or the patient) to communicate with the implantable system 411. In some embodiments, the external system 412 includes a programming device intended for a clinician or other caregiver to initialize and adjust settings for the implantable system 411 and a remote control device intended for use by the patient. For example, the remote control device may allow the patient to turn a therapy on and off and/or adjust certain patient-programmable parameters of the plurality of neurostimulation parameters. The external system 412 may include personal devices such as phones and tablets. The external system may include other processing and/or storage device(s). Some external device examples include a device programmer, a remote control, a phone or tablet, a local system (e.g., local computer, network of computers, network attached storage (NAS), or portable data storage such as a flash drive), or a remote system (e.g., cloud-based systems).

The neurostimulation lead(s) of the lead system 407 may be placed adjacent, i.e., resting near, or upon the dura, adjacent to the spinal cord area to be stimulated. For example, the neurostimulation lead(s) may be implanted along a longitudinal axis of the spinal cord of the patient. Due to the lack of space near the location where the neurostimulation lead(s) exit the spinal column, the implantable neurostimulator 402 may be implanted in a surgically-made pocket either in the abdomen or above the buttocks, or may be implanted in other locations of the patient's body. The lead extension(s) may be used to facilitate the implantation of the implantable neurostimulator 402 away from the exit point of the neurostimulation lead(s).

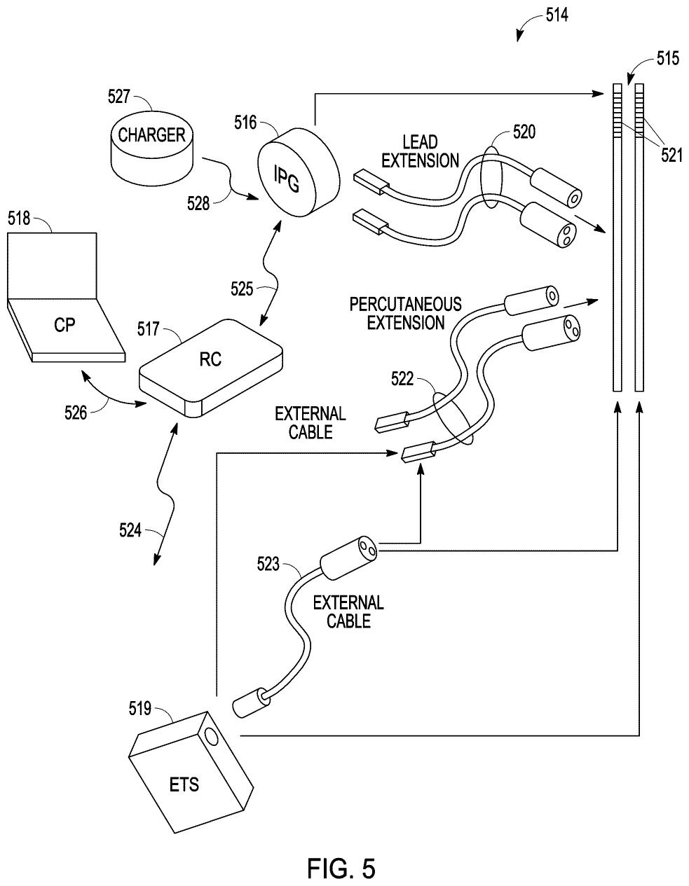

FIG. 5 illustrates, by way of example, an embodiment of a SCS system, which also may be referred to as a Spinal Cord Modulation (SCM) system. A similar system, with DBS lead(s), may be used to provide a DBS system. The SCS system 514 may generally include one or more (illustrated as two) of implantable neurostimulation leads 515, an electrical waveform generator 516 such as an implantable pulse generator, an external remote controller (RC) 517, a clinician's programmer (CP) 518, and an external trial modulator (ETM) 519. IPGs are used herein as an example of the electrical waveform generator. However, it is expressly noted that the waveform generator may be configured to deliver regular, repeating patterns of pulses or in complex patterns that appear to be irregular patterns of pulses where pulses have differing amplitudes, pulse widths, pulse intervals, and bursts with differing number of pulses. It is also expressly noted that the waveform generator may be configured to deliver electrical waveforms other than pulses. The waveform generator 516 may include pulse generation circuitry that delivers electrical neurostimulation energy in the form of a pulsed electrical waveform (i.e., a temporal series of electrical pulses) to the electrode contacts in accordance with a set of neurostimulation parameters. The electrical waveform may include first phases of a first polarity and second phases of a second polarity opposite the first polarity. Neural tissue may be therapeutically stimulated using both the first phases and the second phases of the electrical waveform. The electrical waveform includes a plurality of interphase intervals, each of the plurality of interphase intervals separating individual ones of the first phases and individual ones of the second phases. The second phases may be used to reduce built up charge from the at least one electrode contact caused by the first phases and the first phases may be used to reduce built up charge from the at least one electrode contact caused by the second phases. The waveform generator 516 may be physically connected via one or more percutaneous lead extensions 520 to the neurostimulation leads 515, which carry a plurality of electrode contacts 521. As illustrated, the neurostimulation leads 515 may be percutaneous leads with the electrode contacts arranged in-line along the neurostimulation leads. Any suitable number of neurostimulation leads can be provided, including only one, as long as the number of electrode contacts is greater than two (including the waveform generator case function as a case electrode contact) to allow for lateral steering of the current. Alternatively, a surgical paddle lead can be used in place of one or more of the percutaneous leads.

The ETM 519 may also be physically connected via the percutaneous lead extensions 522 and external cable 523 to the neurostimulation leads 515. The ETM 519 may have similar waveform generation circuitry as the waveform generator 516 to deliver electrical neurostimulation energy to the electrode contacts accordance with a set of neurostimulation parameters. The ETM 519 is a non-implantable device that is used on a trial basis after the neurostimulation leads 515 have been implanted and prior to implantation of the waveform generator 516, to test the responsiveness of the neurostimulation that is to be provided. Functions described herein with respect to the waveform generator 516 can likewise be performed with respect to the ETM 519.

The RC 517 may be used to telemetrically control the ETM 519 via a bi-directional RF communications link 524. The RC 517 may be used to telemetrically control the waveform generator 516 via a bi-directional RF communications link 525. Such control allows the waveform generator 516 to be turned on or off and to be programmed with different neurostimulation parameter sets. The waveform generator 516 may also be operated to modify the programmed neurostimulation parameters to actively control the characteristics of the electrical neurostimulation energy output by the waveform generator 516. A clinician may use the CP 518 to program neurostimulation parameters into the waveform generator 516 and ETM 519 in the operating room and in follow-up sessions. The waveform generator 516 may be implantable. The implantable waveform generator 516 and the ETM 519 may have similar features as discussed with respect to the neurostimulator 202 described with respect to FIG. 2.

The CP 518 may indirectly communicate with the waveform generator 516 or ETM 519, through the RC 517, via an IR communications link 526 or other link. The CP 518 may directly communicate with the waveform generator 516 or ETM 519 via an RF communications link or other link (not shown). The clinician detailed neurostimulation parameters provided by the CP 518 may also be used to program the RC 517, so that the neurostimulation parameters can be subsequently modified by operation of the RC 517 in a stand-alone mode (i.e., without the assistance of the CP 518). Various devices may function as the CP 518. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 518. Alternatively, such programming methodologies can be performed using firmware or hardware. In any event, the CP 518 may actively control the characteristics of the electrical neurostimulation generated by the waveform generator 516 to allow the desired parameters to be determined based on patient feedback, sensor feedback or other feedback and for subsequently programming the waveform generator 516 with the desired neurostimulation parameters. To allow the user to perform these functions, the CP 518 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant waveform generator, implant waveform generator and lead(s), replace waveform generator, replace waveform generator and leads, replace or revise leads, explant, etc.), generate a pain map of the patient, define the configuration and orientation of the leads, initiate and control the electrical neurostimulation energy output by the neurostimulation leads, and select and program the IPG with neurostimulation parameters in both a surgical setting and a clinical setting.

An external charger 527 may be a portable device used to transcutaneously charge the waveform generator via a wireless link such as an inductive link 528. Once the waveform generator has been programmed, and its power source has been charged by the external charger or otherwise replenished, the waveform generator may function as programmed without the RC or CP being present.

The neurostimulation may be based on sensed physiological signals. Examples of such signals may include, but are not limited to, local field potentials, evoked compound action potentials (ECAPs), evoked resonant neural activity (ERNA), cardiac activity (e.g., electrocardiogramuscle activity (e.g., electromyography (EMG)), brain activity (e.g., electroencephalography (EEG)), electroneuronography (ENOG)), galvanic skin responses (GSR), impedance, or movement (e.g., movement detected using an accelerometer or camera). For example, the signals may be used to program the desired neurostimulation parameters and/or may be used to control the timing of the therapy (e.g., when to initiate the therapy, when to suspend the therapy, and/or when and/or how to change the therapy).

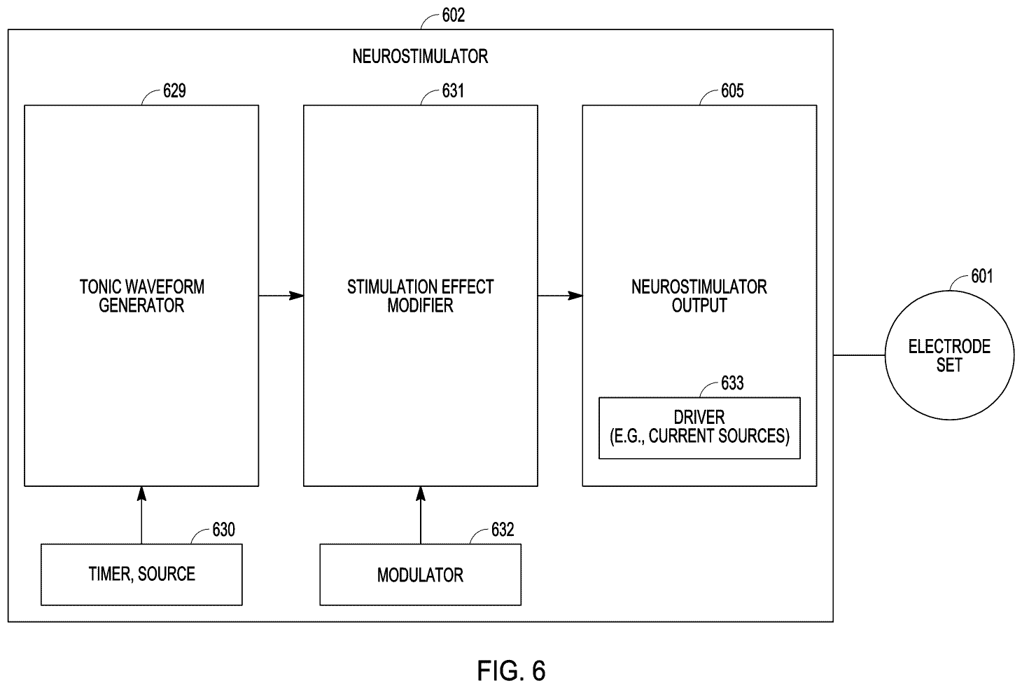

FIG. 6 illustrates, by way of example and not limitation, a neurostimulator embodiment including a tonic waveform generator, a neurostimulation modifier, and a neurostimulator output. The illustrated neurostimulator 602 such as may be implemented in the neurostimulation system 100 of FIG. 1 and may be an example of the neurostimulator 202 in FIG. 2. The neurostimulation control circuit 206 in the neurostimulator of FIG. 2 may include a tonic waveform generator 629 with a timer, source 630, and a stimulation effect modifier 631 with a modulator 632. The neurostimulator output circuit 605 may be an embodiment of the neurostimulator output 205 in the neurostimulator of FIG. 2. The neurostimulator output circuit 605 may include driver circuitry 633 used to deliver the neurostimulation energy to tissue using active electrodes within the electrode set 601. An example of driver circuitry 633 is a current source.

The tonic waveform generator 629 may be configured to generate a train of tonic pulses. The timer/source circuitry 630 may provide the tonic pulse parameters and timing for creating the train of tonic pulses. The tonic pulses may have a uniform pulse-to-pulse interval. The neurostimulation modifier 631 may be configured to apply a neurostimulation-effect modulation, such as provided by the modulator 632, to modify the train of tonic pulses. The neurostimulator output 605 may be configured to use the modified train of pulses to deliver a sequence of electrical pulses to a neural target using the electrode set. The neurostimulation modifier 631 may be configured to apply the neurostimulation-effect modulation 632, which may be a modulation function, to change at least one stimulation parameter for at least a portion of the train of tonic pulses to enhance or reduce a neurostimulation effect for one or more pulses within the sequence of electrical pulses. In some embodiments, the neurostimulation modifier 631 may be implemented using firmware to adjust the current sources. By way of example and not limitation, this may involve adjusting signal inputs to the current sources. In some embodiments, the neurostimulation modifier 631 may be implemented using hardware circuits configured to modulate the train of tonic pulses using the neurostimulation-effect modulation. In some embodiments, the neurostimulation modifier 631 may be implemented using software to process digital tonic pulses into a modified digital train of pulses. By way of example and not limitation, the neurostimulation effect of one or more pulses that may be enhanced or reduced may include providing some pulses within the electrical pulse energy that engage neural tissue and to providing other pulses within the electrical pulse energy that do not engage neural tissue, or providing some pulses within the electrical pulse energy that are supra-threshold stimulation and providing other pulses within the electrical pulse energy that are sub-threshold stimulation. Where the threshold is a perception threshold for the patient, by way of example and not limitation, the patient is able to perceive (e.g., perception such as paresthesia) supra-threshold stimulation and is not able to perceive sub-threshold stimulation. The threshold may correspond to other effect(s)/neural response(s) to the stimulation.

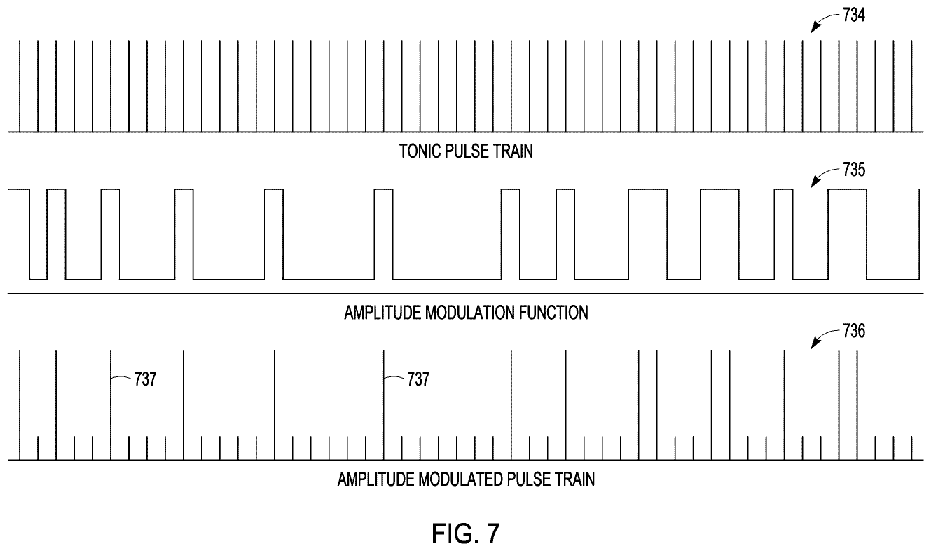

FIG. 7 illustrates, by way of example and not limitation, a method for modifying a train of tonic pulses. A tonic train of pulses 734 may be generated by the tonic waveform generator 629. A modulation function 735, such as the illustrated amplitude modulation function, may be applied by the neurostimulation modifier 631 to the tonic train of pulses to create a modulated pulse train 736. Each bar represents a pulse. The pulse may be monophasic, biphasic or multiphasic with more than two phases. The pulses in the tonic pulse train 734 that are aligned with the pulses of the modulation function will pass (or be enhanced) as illustrated by the larger amplitude pulses in the modulated pulse train 736, and the pulses that are not aligned with the pulses of the modulation function will be bypassed (or be diminished or reduced) with respect to the aligned pulses as illustrated by the smaller amplitude pulses in the modulated pulse train 736. Some embodiments, by way of example and not limitation, the energy from the by-passed or reduced pulses are stored and discharged later to boost the output of other charges. The energy may be stored in energy storage units such as capacitor(s) and/or electro-thermal energy storage (ETES) component(s). The illustrated amplitude modulation function 735 is but other functions or relationships may be used. For example, some embodiments use a charge modulation function, where the function changes the overall charge contribution of the waveform. As illustrated, the larger pulses 737 in the modulated pulse train 736 have a non-tonic arrangement as determined by the modulation function 735. By way of example and not limitation, these larger pulses 737 may be engage neural tissue or cause supra-threshold stimulation. Thus, although the pulse-to-pulse spacing is tonic, the effect of the neural stimulation is not tonic.

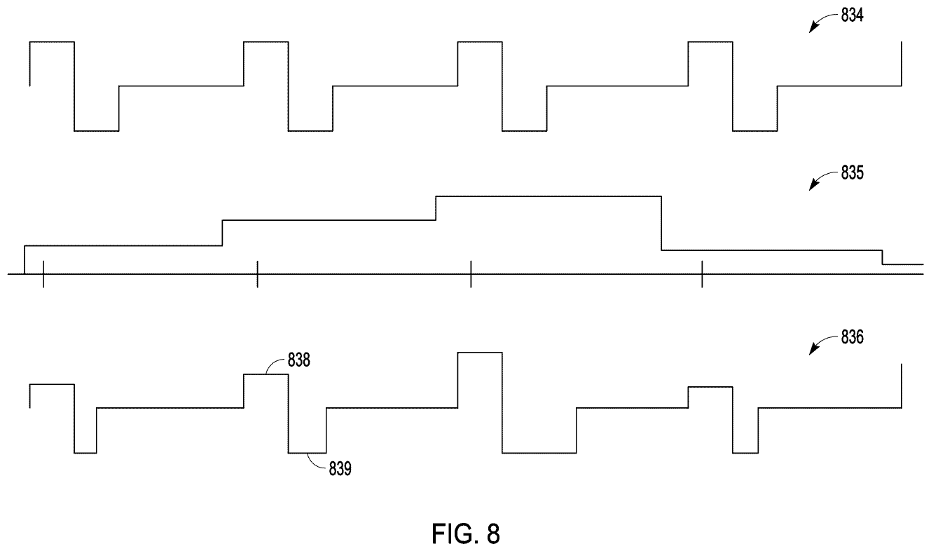

FIG. 8 illustrates, by way of example and not limitation, a tonic waveform 834, a modulation waveform 835 to modulate the tonic waveform 834, and a modulated tonic waveform 836 to provide a sequence of electrical pulses 835. The modulation waveform may be referred to as a charge modulation function, which determines the amount of charge being delivered to the neural tissue at different times. This also may be referred to as a neural stimulation dose modulation function, and a dose of neurostimulation generally corresponds an amount of charge over a period of time. The illustrated tonic waveform includes biphasic pulses including a first phase 838 and a second phase 839. The amplitude modulation function 835 has discrete steps. The resulting modulated pulse train 836 increases or decreases the amplitude of the first phase with the varying magnitude of the amplitude modulation function and increases or decreases the width of the second phase with the varying magnitude of the amplitude modulation function.

This figure illustrates the modulation of two stimulation parameters (e.g., amplitude and width), and further illustrates that a stimulation parameter may be modulated for one phase of a biphasic or multiphasic pulse without having to modulate the same parameter in other phases of the biphasic or the multiphasic pulse. A stimulation parameter may be modulated for one phase of a biphasic or multiphasic pulse and another parameter may be modulated in other phases of the biphasic or the multiphasic pulse, while still providing or at least improving charge balancing. The charge is balanced if the area under the first phase curve is approximately equal to area under the second phase curve. However, in the case if the pulse phases are configured with different areas under the curve, additional charge balancing pulses may be provided intermittently.

The resulting modulated pulse train may be a charge modulated or a combination/hybrid modulation of both amplitude (A) and pulse width (W), the stimulation phase may have amplitude modulation in the leading phase, and pulse width modulation in the charge recovery phase, or vice versa. The amplitude (A) and pulse width (W) may be determined as follows:

$$Am ( tn ) = A ( tn ) * [ Q ( tn ) / w ( t n ) ] * logic ( A ( tn ) > 0 ) + A ( tn ) * logic ( A ( tn ) < 0 ) ; \\ Wm ( n ) = w ( tn ) * logic ( A ( tn ) > 0 ) + w ( tn ) * [ Q ( tn ) / A ( tn ) ] * logic A ( tn ) < 0 ) .$$

Some embodiments may use combinations of two or more of an amplitude modulation function, a charge modulation function and a pulse width modulation. Thus, a hybrid modulation of the following effective combinations may be used: amplitude and rate; amplitude and pulse width; pulse width and rate; amplitude, pulse rate and rate, amplitude; and any of the above plus burst. A ON/OFF cycling control or an envelope modulation function may be applied to achieve bursting or cycling delivery of the above hybrid combination. Various hybrid modulation pulse trains may be concatenated to achieve more complicated modulated pulse sequence.

The charge modulation function in FIG. 8 is illustrated as a step function, by way of example and not limitation. Other modulation functions, such as but not limited to continuous or discrete ramping, trailing, triangular, gaussian, exponential and the like can also be used.

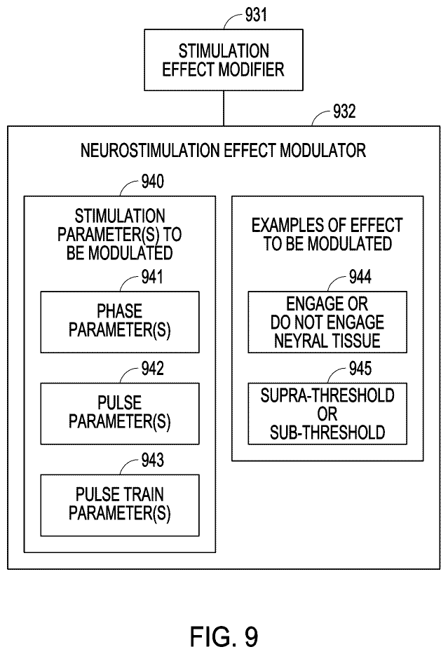

FIG. 9 illustrates, by way of example and not limitation, a neurostimulation effect modifier 931 and corresponding modulator 932 which may be a specific embodiment of the neurostimulation effect modifier 631 and modulator 632 illustrated in FIG. 6. The modulator 932 may be configured to modulate a stimulation parameter 940 such as one or more phase parameter(s) 941, one or more pulse parameter(s) 942, or one or more pulse train parameter(s) 943. The phase parameter(s) 941 may be specific to an individual phase, and may include an amplitude, width, charge or shape, The pulse parameter(s) 942 at least partially define pulses, and may include amplitude, width, charge, phase, shape, and the like. The pulse train parameter(s) 943 may include parameters that at least partially define a train of pulses in the tonic waveform and may include a frequency, a burst on or off duration for the pulse train, or a duty cycle. Some embodiments allow the user to select (e.g., from a list of available parameters) the parameter(s) that is (are) intended to be modified by the modulator and/or select the modulation function. The modulator 932 may be configured to modulate the signal to enhance or diminish an effect of the stimulation pulse. The effect may be whether the pules engages or does not engage the neural tissue 944 or whether the pulses are above a threshold of neural response (e.g., super-perception) or below the threshold of neural response (e.g., sub-perception) 945. The present subject matter is not limited to these effects. Other effects, such as pulses that prime neural tissue or change the excitability of neural tissue, may be used. Some embodiments allow the user to select (e.g., from a list of available effects) the effect that is intended to be modified by the modulator.

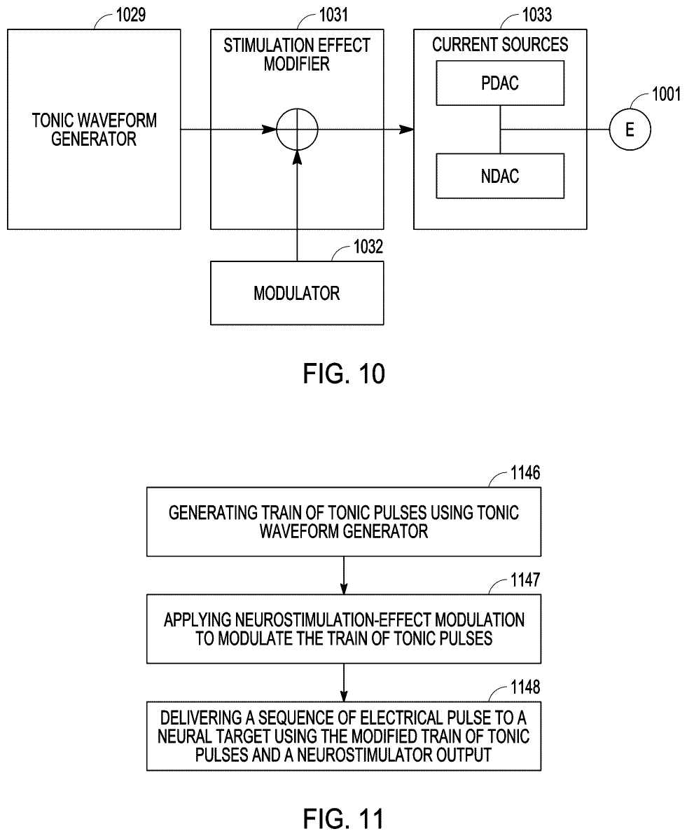

FIG. 10 illustrates, by way of example and not limitation, a neurostimulator embodiment, similar to FIG. 6, including a tonic waveform generator 1029, a neurostimulation modifier 1031, and current sources 1033. It is noted that the current sources are an example of an energy source, and other energy source(s) may be implemented in place of or with the current sources. Examples of other energy source(s) include voltage source(s) and energy storage units such as capacitor(s) and/or electro-thermal energy storage (ETES) component(s). The current sources 1033 may include a plurality of current sources that can be applied to any one or more of the electrodes in the electrodes. In some embodiments, each electrode in the electrode set (only one electrode 1001 being illustrated to simplify the figure) may have a dedicated current source, where each dedicated current source includes a PDAC (positive-type digital-to-analog converter) configured to deliver a positive current to the electrode and an NDAC (negative-type digital-to-analog converter) configured to deliver a negative current to the electrode. The signal received by the current sources control which current sources are active and the amount of current being sourced or sunk through the electrode. Thus, if no current sources are active for an electrode, the electrode is not active. If the NDAC is active, then the electrode is configured as a cathode. If the PDAC is active, then the electrode is configured as an anode. Furthermore, the amplitude and pulse width of the current is also controlled by the signal received at the current sources.

The tonic waveform generator 1029 may be configured to generate a train of tonic pulses. The tonic pulses may have a uniform pulse-to-pulse interval. The neurostimulation modifier 1031 may be configured to apply a neurostimulation-effect modulation, such as provided by the modulator 632, to modify the train of tonic pulses. The current sources receive a configuration of the modified train of pulses, and generates the corresponding sequence of electrical pulses to a neural target using the electrode(s). The stimulation effect modifier may be configured to apply the neurostimulation-effect modulation to change at least one stimulation parameter for at least a portion of the train of tonic pulses to enhance or reduce a neurostimulation effect for one or more pulses within the sequence of electrical pulses. The neurostimulation modifier may be implemented using firmware to adjust the current sources. In some embodiments, the tonic waveform generator and the neurostimulation modifier are implemented using software, and both the train of tonic pulses and the modified train of pulses are digital. The digital pulses in train of tonic pulses can be summed (or other function) to digital pulse modifications to create the modified train of pulses to the current sources. In some embodiments, hardware circuits are used to modulate the train of tonic pulses using the neurostimulation-effect modulation. The neurostimulation-effect modulation provided by the modulator 1032 may be user-programmable. The neurostimulation-effect modulation provided by the modulator 1032 may be a predetermined function programmed in the system.

FIG. 11 illustrates, by way of example and not limitation, a method for modifying a train of tonic pulses. The method may include: generating, using a tonic waveform generator, a train of tonic pulses having a uniform pulse-to-pulse interval 1146; applying, using a neurostimulation modifier, a neurostimulation-effect modulation to modify the train of tonic pulses 1147; and delivering a sequence of electrical pulses to a neural target using an electrode set that includes at least one electrode 1148. The modified train of tonic pulses and a neurostimulator output are used to deliver the sequence. The applied neurostimulation-effect modulation changes at least one stimulation parameter for at least a portion of the train of tonic pulses to enhance or reduce a neurostimulation effect for one or more pulses within the sequence of electrical pulses. A driver may be used to drive the sequence to the electrode set and to the neural tissue. The train of tonic pulses may be modified using firmware, hardware, software, or various combinations thereof.

The applied neurostimulation-effect modulation changes at least one stimulation parameter for at least a portion of the train of tonic pulses to enhance or reduce a neurostimulation effect for one or more pulses within the sequence of electrical pulses. By way of example, some pulses within the electrical pulse energy engage neural tissue and other pulses within the electrical pulse energy that do not engage neural tissue. In an example, some pulses within the electrical pulse energy may correspond to supra-threshold stimulation and other pulses within the electrical pulse energy may correspond to sub-threshold stimulation.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

1. A method, comprising:
generating, using a tonic waveform generator, a train of tonic pulses having a uniform pulse-to-pulse interval;
applying, using a neurostimulation modifier, a neurostimulation-effect modulation to modify the train of tonic pulses; and
delivering a sequence of electrical pulses to a neural target using an electrode set that includes at least one electrode, wherein the modified train of tonic pulses and a neurostimulator output are used to deliver the sequence,
wherein the applied neurostimulation-effect modulation changes at least one stimulation parameter for at least a portion of the train of tonic pulses to enhance or reduce a neurostimulation effect for one or more pulses within the sequence of electrical pulses.

2. The method of claim 1, the sequence is delivered using a driver to drive the sequence to the neural target.

3. The method of claim 2, wherein the driver includes energy sources, and wherein the neurostimulation-effect modulation is applied using firmware to adjust energy sources.

4. The method of claim 2, wherein the neurostimulation-effect modulation is applied using hardware circuits configured to modulate the train of tonic pulses.

5. The method of claim 2, wherein the tonic waveform generator and the neurostimulation modifier are implemented using software, and both the train of tonic pulses and the modified train of pulses are digital.

6. The method of claim 1, wherein the neurostimulation-effect modulation is a charge modulation function.

7. The method of claim 6, wherein the charge modulation function is applied by modulating more than one stimulation parameter for at least the portion of the train of tonic pulses.

8. The method of claim 7, wherein the charge modulation function is applied by modulating at least two stimulation parameters from: amplitude, pulse width, pulse frequency, a charge balance configuration, burst duration, burst frequency or burst ON/OFF duty cycle.

9. The method of claim 1, further comprising receiving a user input to program the neurostimulation-effect modulation.

10. The method of claim 1, wherein the neurostimulation-effect modulation is a predetermined function that is not user-programmable.

11. The method of claim 1, wherein the sequence of electrical pulses includes a section of monophasic pulses and a recharge balance section for balancing delivered net charge.

12. The method of claim 1, wherein the sequence of electrical pulses includes two or more phases.

13. The method of claim 12, wherein the neurostimulation-effect modulation is applied by providing a first value for at least one parameter in a first phase independent of a second value for the at least one parameter in a second phase.

14. The method of claim 1, wherein the neurostimulation-effect modulation is applied to provide some pulses within the sequence of electrical pulses that engage neural tissue and to provide other pulses within the sequence of electrical pulses that do not engage neural tissue.

15. The method of claim 1, wherein the neurostimulation-effect modulation is applied to provide some pulses within the sequence of electrical pulses that are supra-threshold stimulation and to provide other pulses within the sequence of electrical pulses that are sub-threshold stimulation.

16. A non-transitory machine-readable medium including instructions, which when executed by a machine, cause the machine to perform a method comprising:
generating, using a tonic waveform generator, a train of tonic pulses having a uniform pulse-to-pulse interval;
applying, using a neurostimulation modifier, a neurostimulation-effect modulation to modify the train of tonic pulses; and
delivering a sequence of electrical pulses to a neural target using an electrode set that includes at least one electrode, wherein the modified train of tonic pulses and a neurostimulator output are used to deliver the sequence,
wherein the applied neurostimulation-effect modulation changes at least one stimulation parameter for at least a portion of the train of tonic pulses to enhance or reduce a neurostimulation effect for one or more pulses within the sequence of electrical pulses.

17. The non-transitory machine-readable of claim 16, wherein the sequence of electrical pulses includes a section of monophasic pulses and a recharge balance section for balancing delivered net charge.

18. A system, comprising:
an electrode set including at least one electrode;
a neurostimulator, including:
a tonic waveform generator configured to generate a train of tonic pulses, wherein the tonic pulses have a uniform pulse-to-pulse interval;
a neurostimulation modifier configured to apply a neurostimulation-effect modulation to modify the train of tonic pulses; and
a neurostimulator output configured to use the modified train of tonic pulses to deliver a sequence of electrical pulses to a neural target using the electrode set,
wherein the neurostimulation modifier is configured to apply the neurostimulation-effect modulation to change at least one stimulation parameter for at least a portion of the train of tonic pulses to enhance or reduce a neurostimulation effect for one or more pulses within the sequence of electrical pulses.

19. The system of claim 18, wherein the neurostimulation-effect modulation is user-programmable.

20. The system of claim 18, wherein the neurostimulation-effect modulation is a predetermined function programmed in the system.
